(19) United States
(12) Patent Application Publication
(10) Pub. No.: US 2003/0003131 A1
(43) Pub. Date: Jan. 2, 2003

(54) Method for manufacture of free-flowing powder containing water-dispersible sterols

(76) Inventors: Matthew Dyer, Mt. Zion, IL (US); Brent Flickinger, Decatur, IL (US); Thomas Gottemoller, Mt. Zion, IL (US); Brian Yager, Decatur, IL (US)

Correspondence Address: Michael E. Yates, Esq., Archer Daniels Midland Company, 4666 Faries Parkway, Decatur, IL, 62526, US

(21) Appl. No.: 10/163,379

(22) Filed: Jun. 7, 2002

Related U.S. Application Data
(60) Provisional application No. 60/300,281, filed on Jun. 22, 2001.

(51) Int. Cl. A61K 47/00; A61K 31/56; B29B 9/00

(52) U.S. Cl. 424/439; 264/5; 514/169

(57) ABSTRACT

A process for producing a free-flowing powder comprising water dispersible sterols, the process comprising commingling sterols and lecithin in an organic solvent, removing the solvent to produce a commingled solid material, grinding the commingled solid to produce a powder, hydrating the powder in water, adding a spray drying adjunct before or after homogenization of the powder, and spray drying the homogenized product.

This application claims priority to provisional application Ser. No. 60/300,281, filed on Jun. 22, 2001.

### FIELD OF THE INVENTION

This invention relates to a composition and method for reducing cholesterol absorption and serum cholesterol in humans. It represents an improvement in that the material also contains a spray drying adjunct such as maltodextrin.

### BACKGROUND OF THE INVENTION

Phytosterols are plant sterols structurally similar to cholesterol that have been known for many years to reduce cholesterol absorption and serum cholesterol levels while not being absorbed themselves. Lowering of circulating cholesterol and low density lipoprotein cholesterol is an important part of a strategy to prevent and treat cardiovascular disease and especially coronary heart disease. Cholesterol absorption is a critical component of whole body cholesterol metabolism. Cholesterol derived from the diet and also from endogenous biliary secretion enters the intestine, and approximately 50% of the mixed intestinal load is absorbed, Bosner, M. S., Ostlund, R. E., Jr., Osofisan, O., Grosklos, J., Fritschle, C., Lange, L. G 1993. The failure to absorb cholesterol quantitatively is therefore a key mechanism for the elimination of cholesterol from the body.

Drugs commonly used to treat high cholesterol levels have little or no effect on cholesterol absorption. For example, the potent new hydroxymethylglutaryl coenzyme A reductase inhibitors have a primary action to reduce cholesterol synthesis rather than increase cholesterol elimination. Bile acid sequestrants such as the ion:exchange resin cholestyramine act within the intestine but do not bind cholesterol and may actually increase cholesterol absorption when given chronically. McNamara, D. J., N. O. Davidson, P. Samuel, and E. H. Ahrens, Jr. 1980, Cholesterol absorption in man: effect of administration of clofibrate and/or cholestyramine. J. Lipid Res. 21:1058-1064. Although orally-administered neomycin reduces cholesterol absorption effectively, it is toxic and has the disadvantage of requiring chronic administration of a potent antibiotic, Samuel, P. 1979. Treatment of hypercholesterolemia with neomycinatime for reappraisal. N. Engl. J. Med. 301:595597. The drug Cytellin.RTM., an aqueous suspension of mixed phytosterols, was produced by Eli Lilly Co. for treatment of elevated cholesterol, but it has not been sold since 1985. As seen, it is apparent that new inhibitors of cholesterol absorption would complement currently-available treatment for high serum cholesterol.

Since phytosterols are natural products which are non-toxic and byproducts of food processing, they may be important in the treatment of individuals with mildly-increased serum cholesterol, or for the general population in food products or dietary supplements. The use of phytosterols could reduce the need for systematically-absorbed drugs.

Despite their potential attractiveness, the usefulness of phytosterols has been limited by small and erratic effectiveness and a large dosage requirement. For example, doses of 5-18 g sitosterol/day reduced serum cholesterol by 16-20%. Farquhar, J. W. and M. Sokolow, 1958. A dose-response study showed that 3-9 g/day of powdered sitosterol was needed to decrease serum cholesterol levels by 12%. Lees, A. M., H. Y. I. Mok, R. S. Lees, M. A. McCluskey, and S. M. Grundy. 1977. Plant sterols as cholesterol lowering agents: clinical trials in patents with hypercholesterolemia and studies of sterol balance, Atherosclerosis 28:325-338. To reduce the amount needed, recent experiments have used sitostanol instead of sitosterol because it appears to be more potent than other phytosterols and is non-absorbable, Sugano, J., H. Morioka, and I. Ikeda. (1977) A comparison of hypocholesterolemic activity of .beta.-sitosterol and .beta.sitostanol in rats. J. Nutr. 107:2011-2019. In subjects with severe hypercholesterolemia sitostanol at 1.5 g/day reduced serum cholesterol by 15%, Heinemann, T., O. Leiss, and K. von Bergmann (1986) Effect of low dose sitostanol on serum cholesterol in patients with hypercholesterolemia. Atherosclerosis 61:219-223. However, sitostanol at 3 g/day had no effect in subjects with moderate hypercholesterolemia. Denke, M. A. (1995), Lack of efficacy of low dose sitostanol therapy as an adjunct to a cholesterol-lowering diet in men with moderate hypercholesterolemia. Am. J. Clin. Nutr. 61:392-396.

Several investigators have proposed ways to increase the solubility or bioavailability of phytosterols in order to make them more useful. Based on studies in rats and the finding that phytosterol esters are much more soluble in oil than the free sterols, it has been proposed to use phytosterol esters in oil to lower cholesterol absorption, Mattson, F. H., R. A. Volpenhein, and B. A. Erickson (1977), Effect of plant sterol esters on the absorption of dietary cholesterol. J. Nutr. 107:11391146. U.S. Pat. No. 5,502,045 describes the use of sitostanol ester in oil for the treatment of hypercholesterolemia in humans. It was found that 2.8 g sitostanol/day given as sitostanol ester in margarine reduced LDL cholesterol by 16%. Miettinen, T. A., P. Puska, H. Gylling, H. Vanhanen, and E. Vartiainen (1995), Reduction of serum cholesterol with sitostanolester margarine in a mildly hypercholesterolemic population. N. England J. Med. 333:13081312. However, the use of sitostanol ester dissolved in dietary fat has the disadvantage of requiring the administration of 2350 g/day of dietary fat and of being 21% less effective at reducing cholesterol absorption in humans compared to the unesterified sterol. Mattson, F. H., S. M. Grundy, and J. R. Crouse, (1982), Optimizing the effect of plant sterols on cholesterol absorption in man. Am. J. Clin. Nutr. 35:697-700.

Other workers have investigated ways to improve the usefulness of unesterified phytosterols. in International Patent Publication WO 95/00158 a complex of sitosterol and the unabsorbable dietary fiber pectin reduced serum cholesterol by 16.4% when given to hypercholesterolemic humans in a dose of 2.1 g/day. However, no measurements of an effect on cholesterol absorption were made, and the complex was only about 50% soluble even at strongly alkaline pH, suggesting that the bioavailability of the sitosterol component was limited.

U.S. Pat. No. 5,244,887 describes the use of stanols including sitostanol in food additives to reduce cholesterol absorption. In U.S. Pat. No. 5,244,887, for preparation of the additives, sitostanol is dissolved with an edible solubilizing agent such as triglyceride, an antioxidant such as tocopherol, and a dispersant such as lecithin, polysorbate 80, or sodium lauryl sulfate. However, no data were given to guide one in the selection of the most effective components and their amounts or specific methods of preparation. Effectiveness in reducing cholesterol absorption was also not determined. The preferred embodiment consisted of 25% by weight stanols in vegetable oil, but the solubility of sterols in oil is only 2%.

U.S. Pat. No. 5,118,671 describes the production of sitosterol-lecithin complexes for pharmaceutical use but does not consider oral use for cholesterol lowering.

Cholesterol is absorbed from an intestinal micellar phase containing bile salts and phospholipids which is in equilibrium with an oil phase inside the intestine. Delivery of phytosterol as a solid powder or aqueous suspension is not preferred because of the limited rate and extent of solubility in intestinal liquid phases. Esterification of the phytosterol with delivery through the oil phase of foods is an alternative route but has the disadvantage of use of edible oils as the carrier.

U.S. Pat. Nos. 5,932,562 and 6,063,776 provide a delivery system for plant sterols, particularly sitostanol, which avoids an oil phase and which provides bioavailable sitostanol at a level which reduces cholesterol absorption as much as 37%, while at the same time using an excellent taste emulsifier in as low amounts as possible.

U.S. Pat. Nos. 5,932,562 and 6,063,776 also provide a water soluble composition which provides the sitostanol, not dissolved in fat, but rather combined with a preferred emulsifier (Sodium Stearoyl 2-lactylate) (SSL) in an aqueous vesicular complex which can enter directly into the intestinal micellar phase and is therefore highly bioavailable.

U.S. Pat. Nos. 5,932,562 and 6,063,776 also provide a composition of preferred enhanced solubility that contains a plant sterol, preferably sitostanol mixed with an emulsifier even better than phospholipids, namely SSL, which has water solubility in excess of 90%.

U.S. Pat. Nos. 5,932,562 and 6,063,776 also provide a method for reducing cholesterol absorption from food products containing cholesterol by mixing finely divided water soluble powder of an aqueous homogeneous micellar mix of sitostanol and SSL with a food product which is to be ingested.

U.S. Pat. Nos. 5,932,562 and 6,063,776 also provide a method of manufacturing a dry, finely divided water soluble powder which contains a plant sterol, preferably sitostanol, and lecithin, which is highly water soluble, so that when in contact with an aqueous system it will provide an aqueous vesicular complex which can enter directly into the intestinal micellar phase to inhibit cholesterol absorption.

It is an objective of the present invention to provide improved processing and other characteristics to the composition of U.S. Pat. Nos. 5,932,562 and 6,063,776 by the addition of a spray drying adjunct such as starches or hydrolyzed starches, and in a preferred embodiment maltodextrin, which provides additional benefits of improving the characteristics of the sterol/lecithin composition in food and beverage applications.

The method and manner of achieving each of the above objectives, as well as others, will become apparent from the detailed description of the invention which follows hereinafter.

### SUMMARY OF THE INVENTION

A composition for inhibiting cholesterol absorption from the intestine is described. The composition comprises phytosterols, preferably sitostanol, dispersed in an aqueous base emulsifier, preferably lecithin or SSL. The mole ratio of sterol to emulsifier should be 1:0.1 to 1:10, preferably 1:0.9 to 1:0.5.

The phytosterol-emulsifier complex is prepared by high shear mixing, for example by vortexing, mixing, sonicating or passing through a small orifice of a phytosterol: emulsifier mixture in water. The dispersed material is then either used as is or dried, for example, by lyophilization or spray-drying. The complex can be used in liquid form prior to any drying, or it can be dried as indicated, and then on contact with liquid it again forms an aqueous vesicular complex which can enter directly into the intestinal micellar phase. No fat is used as a carrier, and surprisingly the system, even when dried, does not change its physical structure from the micelles that contain vesicles, the majority of which contain some plant sterol and some lecithin. In particular, in the present invention, a spray drying adjunct, for example maltodextrin, is added to improve the processing and application performance characteristics of the sterol/lecithin composition.

### DETAILED DESCRIPTION OF THE INVENTION

As previously mentioned, the current invention differs from prior art uses of plant sterols and sitostanol in significant ways.

First, the dose needed to reduce cholesterol absorption is lower than previously reported, namely 25-300 mg of sitostanol. Second, the preferred formulation does not contain triglycerides or oils. The phytosterol is not dissolved in fat, but rather is combined with phospholipid to form an aqueous vesicular complex which can enter directly into the intestinal micelle phase. Third, the mix can be prepared in solid form by drying an aqueous sitostanol/emulsifier vesicular formulation with retention of solubility in artificial bile. Fourth, the mix is effective when consumed separately from cholesterol-containing foods. Fifth, the mix can be added to non-cholesterol containing and fat-free foods and beverages. Sixth, the mix is prepared in a manner to prevent self association of sitostanol as occurs when it is dried from organic solvents containing sitostanol and solubilizing agents. The mix herein referenced has the advantage of a high degree of bioavailability as assayed with artificial bile in vitro. This is significant and something that cannot be achieved with fat carrier systems.

The composition is useful for reducing cholesterol absorption in humans at doses between 10 and 1000 mg, and a preferred dose is 100-300 mg. The dose is less than required by current protocols. The composition may be used in capsule or tablet form as a drug or dietary supplement. Alternatively, it may be used in foods as a food additive or substance generally recognized as safe for human consumption.

In preparation of the composition useful for reducing cholesterol in highly bioavailable form, the first step is to provide an aqueous homogeneous micellar mix of the plant sterol with the preferred emulsifier of choice.

One preferred method is to use sitostanol because only small amounts are absorbed in the small intestine, but on the other hand, this plant sterol shows high inhibition of cholesterol absorption. Similar compounds are also suitable, including sitosterol, campesterol, stigmasterol. Moreover, lignans, such as sesamin, and saponins are also useful for this purpose, but sitostanol is preferred. Sterol esters may also be used.

The preferred phospholipid for the present invention is lecithin, with an alternative phospholipid system useful to enhance the bioavailability being a mix of lecithin and lysolecithin. Where the mix was used, it was preferred that the mole ratio of lecithin to lysolecithin be at least 1:0.2, preferably 1:0.5.

In this first step, the aqueous homogeneous mixture of the plant sterol and the emulsifier are homogeneously mixed to provide a micellar mix. The preferred mixing form is a high shear mixing. By way of example, vortexing, sonicating, passing through a small orifice such as a French press or other mixing means may be employed. The most preferred mixing is sonication. This disperses the material and enhances the formation of a micellar mix that contains vesicles, the majority of which contain some plant sterol and some emulsifier.

Generally, with respect to sonication, any method that is commonly used for preparation of emulsions can be used to prepare homogeneous mixtures of the plant sterol and the emulsifier, either alone or in combination. For example, Waring blenders, or other high shear mixers can provide acceptable results. Microfluidizers can be used. In this latter procedure, the plant sterol and the emulsifier are forced through ceramic capillaries under high pressure. Where the preferred sonication technique is used, a time within the range of 1.5 minutes to about 4 minutes for sonication is sufficient. On small scale experiments, sonication is typically performed in about 1.5 minutes. Mixers, homogenizers, grinders and spray dryers of various makes and models are well known in the art, as are organic solvents such as hexane.

The drying process is not critical, so long as it does not destroy the vesicular complex formed between the plant sterol and the emulsifier. Generally, non-drastic drying procedures are preferred such as vacuum drying, freeze drying or low-temperature embient air drying. Where heat is employed, the temperature at atmospheric conditions should not exceed 0.degree. C.

As earlier explained, the dosage of the dry powder may be within the range of 10 to 1000 mg per day, and a preferred dose being 25 to 300 mg per day. The most preferred doses to achieve significant cholesterol absorption reduction levels are achieved at a dose range of from 100 mg to 300 mg one to four times daily.

Various permutations of the method and composition of the present invention are presented:

1. COMINGLE sterols and lecithin in organic solvent.

REMOVE solvent to produce solid commingled material.

GRIND solid to produce powder.

HYDRATE powder in water (>140F) and addition of suitable spray drying adjunct with vigorous mixing.

HOMOGENIZE at >3000 psi to produce sterol-lecithin micelle.

SPRAY DRY to produce free-flowing powder.

2. COMINGLE sterols and lecithin in organic solvent.

REMOVE solvent to produce solid commingled material.

GRIND solid to produce powder.

HYDRATE powder in water (>140F) with vigorous mixing.

HOMOGENIZE at >3000 psi to produce sterol-lecithin micelle.

ADDITION of suitable spray drying adjunct.

SPRAY DRY to produce free-flowing powder.

3. COMINGLE sterols and lecithin in organic solvent.

REMOVE solvent to produce granular or powdered commingled material.

HYDRATE powder in water (>140F) and addition of suitable spray drying adjunct with vigorous mixing.

HOMOGENIZE at >3000 psi to produce sterol-lecithin micelle.

SPRAY DRY to produce free-flowing powder.

4. COMINGLE sterols and lecithin in organic solvent.

REMOVE solvent to produce granular or powdered commingled material.

HYDRATE powder in water (>140F) with vigorous mixing.

HOMOGENIZE at >3000 psi to produce sterol-lecithin micelle.

ADDITION of suitable spray drying adjunct.

SPRAY DRY to produce free-flowing powder.

Pasteurization may be added between HOMOGENIZE and SPRAY DRY for 1 & 2, and between HOMOGENIZE and ADDITION for 3 & 4.

The following examples are offered to further illustrate, but not limit the process of the present invention.

### EXAMPLES

1) Sterols were added to lecithin in hexane. The sterols-lecithin mix were applied to a drum dryer. A solid material of commingled sterols-lecithin was scraped from the drum. The solid was ground to a course powder. The course powder was hydrated in water followed by the addition of maltodextrin (>140F; 1.5 parts course powder, 1.5 parts spray drying adjunct; 7 parts water) under vigorous mixing. The was passed solution through a homogenizer at 8000 psi followed by a High Temperature Short Time pasteurizer set at commonly practiced industry settings. Finally, the solution was spray dried using commonly practiced industry settings to produce a free-flowing powder.

2) Sterols were added to lecithin in hexane. The sterols-lecithin mix were applied to a drum dryer. A solid material of commingled sterols-lecithin was scraped from the drum. The solid was ground to a course powder. The course powder was hydrated in water followed (>140F; 1.5 parts course powder, 7 parts water) under vigorous mixing. The solution was passed through a homogenizer at 8000 psi followed by a High Temperature Short Time pasteurizer set a commonly practiced industry settings. Next, 1.5 parts maltodextrin were added under vigorous stirring. The solution was spray dried using commonly practiced industry settings to produce a free-flowing powder. For low pH applications in solution, various gums, such as guar, xanthan, or pectin may be useful as stabilizer.

3) Yogurt products of the formulations shown in Appendix A were made using the compositions of the present invention. As will be recognized by those of skill in the art, any appropriate yogurt culture, and any number of substitute ingredients, may be used in such yogurt product formulations.

It can be seen from the above examples that the composition prepared in accordance with the process of this invention will have improved characteristics for production, processing, handling and applications, and that in general all of the objectives of the invention are achieved.

It should be understood that certain modifications should be and will be apparent to those of ordinary skill in the art, and that such modifications to the precise procedures in compositions set forth herein are intended to come within the spirit and scope of the invention either literally or by doctrine of equivalents. In this light, the following claims are asserted. All references cited herein are hereby incorporated herein by reference in their entirety.

What is claimed is:

1. A process for producing a free-flowing powder comprising water dispersible sterols, the process comprising commingling sterols and lecithin in an organic solvent, removing the solvent to produce a commingled solid material, grinding the commingled solid to produce a powder, hydrating the powder in water, adding a spray drying adjunct before or after homogenization of the powder, and spray drying the homogenized product.

2. The product of the process of claim 1.

3. The product of claim 2, wherein the product is incorporated into a food or beverage product.

4. The food or beverage product of claim 3, wherein the product is a yogurt product.
